(19) United States
(12) Patent Application Publication
(10) Pub. No.: US 2017/0020404 A1
(43) Pub. Date: Jan. 26, 2017

(54) NEUROMONITORING PROBE SYSTEMS AND METHODS

(71) Applicant: Gordon D. Donald, III, Eatontown, NJ (US)

(72) Inventor: Gordon D. Donald, III, Eatontown, NJ (US)

(21) Appl. No.: 15/301,176

(22) PCT Filed: Mar. 30, 2015
(86) PCT No.: PCT/US15/23392

§ 371 Date: Sep. 30, 2016

Related U.S. Application Data
(60) Provisional application No. 61/972,740, filed on Mar. 31, 2014.

(51) Int. Cl. A61B 5/04; A61B 5/00; A61B 5/0492

(52) U.S. Cl. CPC A61B 5/04001; A61B 5/0492; A61B 5/6848

(57) ABSTRACT

A neuromonitoring system comprises a plurality of needle electrodes, a ground electrode, a monopolar stimulating probe having an electrode tip and configured to be coupled to a cable, and a stimulating probe sleeve. The sleeve is adapted and configured to be removably coupled to the monopolar stimulating probe. The sleeve has a slot and is configured and adapted to coaxially slide relative to the monopolar stimulating probe between a first retracted position and a second advanced position. The electrode tip generates an omnidirectional signal when in use while the sleeve is in the retracted position or in an uncoupled position. The electrode tip generates a unidirectional signal when in use while the sleeve is in the advanced position.

### CROSS-REFERENCE TO RELATED APPLICATIONS

Any and all applications for which a foreign or domestic priority claim is identified in the Application Data Sheet as filed with the present application, are hereby incorporated by reference under 37 CFR 1.57. This application claims priority benefit of U.S. Provisional Application No. 61/972,740, filed Mar. 31, 2014, the entirety of which is hereby incorporated by reference herein.

### BACKGROUND OF THE DISCLOSURE

Field of the Disclosure

The present application relates to devices, systems and methods for treating the spine. In certain embodiments, the present application relates to devices, systems and methods for providing neuromonitoring devices, systems and methods for use in connection with spinal stabilization, such as a spinal fusion. In particular, certain embodiments relate to minimally invasive devices, systems and methods for delivering fixation devices and implants into the spine in connection with the use of neuromonitoring devices, systems and methods.

Description of the Related Art

Referencing a lateral surgical access approach may include using one or more of the following surgical instruments: neuromonitoring probe, small dilators, larger dilators, and/or a retractor. After an incision is created, dilators may be used to create a surgical access site that may be followed by the use of a retractor or other specialized tools to create a surgical access corridor.

In a lateral approach to a patient's spine, a psoas muscle, located on either side of the spine, may be separated in order to access the spine and, in particular, an intervertebral disc space or one or more vertebral bodies within a patient's spinal column. Generally, a surgeon tries to avoid nerves of the lumbar plexus that lie within the psoas muscle during such procedures. The anterior third of the psoas muscle is typically considered a safe zone for muscle separation.

To avoid nerves, surgeons may map the position of the nerves near the psoas muscle using neuromonitoring instruments, such as neuromonitoring probes and/or neuromonitoring dilators. The neural elements or nerves of the psoas muscle may be mapped using a stimulating probe. In this manner, the most posterior neural or nerve free area of the psoas muscle may be located and identified. The stimulating probe may then be inserted through the psoas muscle via the most posterior neural or nerve free tissue area or through nearly any other region that is free of neural elements or nerves and toward the spine or into the intervertebral disc space in order to initiate safe tissue separation of the psoas muscle. Dilators are next placed over the probe to create and enlarge a surgical access site. Following the use of dilators, a retractor or other specialized tools are used to further enlarge the surgical access corridor.

### SUMMARY OF THE DISCLOSURE

Various embodiments described herein relate to a neuromonitoring access system that can be used as part of a minimally disruptive approach to the spine. In some embodiments, a minimally invasive surgical system for treating the spine can include at least one neuromonitoring probe assembly.

According to some embodiments, a neuromonitoring system comprises a plurality of needle electrodes, a ground electrode, a monopolar stimulating probe having an electrode tip and configured to be coupled to a cable, and a stimulating probe sleeve. The sleeve is adapted and configured to be removably coupled to the monopolar stimulating probe. The sleeve has a slot and is configured and adapted to coaxially slide relative to the monopolar stimulating probe between a first retracted position and a second advanced position. The electrode tip generates an omnidirectional signal when in use while the sleeve is in the retracted position or in an uncoupled position. The electrode tip generates a unidirectional signal when in use while the sleeve is in the advanced position.

According to some embodiments, the system comprises a slot extending an entire length of the sleeve from a proximal portion to a distal portion. According to some embodiments, the system comprises a slot extending a partial length of the sleeve. According to some embodiments, the sleeve is rotatable about a longitudinal axis of the stimulating probe when coupled. According to some embodiments, the sleeve has a tapered shape. According to some embodiments, the probe is disposable. According to some embodiments, the sleeve is disposable. According to some embodiments, the sleeve conforms to a curvature of the stimulating probe. According to some embodiments, the stimulating probe is adapted and configured to move coaxially relative to retractor blades forming a channel about an axis. According to some embodiments, the sleeve is adapted and configured to move coaxially relative to retractors blades forming a channel about an axis.

According to some embodiments, a method of using a neuromonitoring system comprises providing a monopolar stimulating probe having an electrode tip and a stimulating probe sleeve having a slot. The sleeve is removably coupled to the probe. The sleeve is slid coaxially relative to the probe to selectively shield an electrode tip of the probe. The sleeve can be coupled to the probe prior to insertion of the probe in a patient. The sleeve can be coupled to the probe after insertion of the probe in a patient.

### BRIEF DESCRIPTION OF THE DRAWINGS

FIGS. 1-6 are perspective views of embodiments of at least a portion of a neuromonitoring probe assembly system.

### DETAILED DESCRIPTION OF THE PREFERRED EMBODIMENTS

The neuromonitoring devices, systems and methods disclosed herein have been developed to provide surgeons with the tools and instrumentation for a minimally invasive lateral approach to the lumbar spine. To establish a surgical corridor to the lumbar spine the surgeon bluntly dissects through the external oblique, internal oblique and transversus muscles before exposing the lateral aspect of the psoas muscle. Iatrogenic injury to the sympathetic nerve chain, the nerve roots, the lumbar plexus or individual nerves is most likely to occur during the blunt dissection through the psoas muscle. Neural injury during this surgery can be caused by compression, stretch, transection, hematoma in the psoas and ischemia.

According to some embodiments, a neuromonitoring system comprises one or more, and preferably up to 8 or more, EO sterile twisted pair needle electrodes. According to some embodiments, a neuromonitoring system comprises one or more, and preferably at least two EO sterile single needle electrodes. According to some embodiments, a neuromonitoring system comprises one or more non-sterile sticky pad ground electrodes. According to some embodiments, a neuromonitoring system comprises one or more disposable and/or reusable monopolar stimulating probes with a touchproof cable and/or another suitable cable. According to some embodiments, a neuromonitoring system comprises one or more stimulating probe sleeves.

According to some embodiments, the one or more stimulating probe sleeves comprises a proximal portion and a distal portion. The distal portion preferably defines a distal sleeve opening. The proximal portion preferably defines a proximal sleeve opening. According to some embodiments, the one or more stimulating probe sleeves comprises one or more slots. The slot can extend an entire length of the sleeve from the proximal portion to the distal portion in some embodiments. The slot can extend a partial length of the sleeve in some embodiments. In some embodiments, another opening, such as a hole, can be used in addition to or rather than a slot.

The one or more stimulating probe sleeves can be configured and adapted to be releasably coupled to the one or more monopolar stimulating probes. In some embodiments, the sleeve is configured to be coupled to the stimulating probe prior to probe insertion of the probe into a patient. In some embodiments, the sleeve is configured to be coupled to the stimulating probe after insertion of the probe into a patient. The sleeve is preferably configured and adapted to be positioned generally coaxially with the stimulating probe when coupled. The sleeve is preferably slidable longitudinally along the stimulating probe when coupled. The sleeve is preferably rotatable about the longitudinal axis of the stimulating probe when coupled. For example, the slot in the sleeve can rotate relative to the stimulating probe shaft.

In some embodiments, the stimulating probe preferably comprises an omnidirectional monopoloar electrode tip. At least one advantage of embodiments comprising a sliding, rotating, removable, slotted sleeve is that in use, the probe can be initially advanced and provide electrical stimulation and/or a signal in an omnidirectional fashion with the sleeve detached and/or held back in a withdrawn position. After using the probe in an omnidirectional fashion to map and/or identify possible nerve locations, a particular path can preferably be identified. The sleeve can then be coupled to the probe, if not already coupled, and can be advanced distally toward the surgical location until the sleeve advances to the at least partially cover the omnidirectional stimulating probe tip. The slotted sleeve preferably blocks at least at least portion of the distribution of electrical stimulation and/or signal produced by the stimulating probe, while at least a portion of the distribution of electrical stimulation and/or signal is not blocked, but passes through a slot in the sleeve. Accordingly, use of a sliding, rotating, removable, slotted sleeve can be advantageous to effectively convert the omnidirectional signal of the monopolar electrode tip into a unidirectional signal. It can be advantageous to use the probe in a unidirectional fashion to further navigate the probe and/or identify possible nerve locations along a particular path. The surgeon can preferably rotate the sleeve to orient the slot to control the desired direction of the unidirectional signal.

In some embodiments, the sleeve can be coupled with the probe prior to probe insertion in a patient and/or after probe insertion in a patient. In some embodiments, the sleeve can be coupled with the probe by sliding the sleeve longitudinally proximally along the probe shaft prior to insertion of the probe in a patient. In some embodiments, the sleeve can be coupled with the probe by positioning the probe through the slot of the sleeve. For example, in some embodiments, portions of the sleeve can deflect open and then snap around the shaft of the probe as the slot in the sleeve is forced open and around the probe shaft. In some other embodiments, the sleeve can be wrapped around the probe shaft. Any suitable manner for coupling the slotted sleeve with the probe shaft can be used.

In some embodiments, sleeves can have different dimensions and/or configurations. In some embodiments, sleeves can have different shapes and/or sizes. In some embodiments, the sleeve can have a wall thickness defining a lumen and/or channel through the sleeve. The lumen of some sleeves can approximate the size of the probe shaft. The lumen of some sleeves can be greater than the size of the probe shaft. The lumen of some sleeves can be smaller than the size of the probe shaft. In some embodiments, a diameter of the lumen can be constant along the longitudinal axis. In some embodiments, a diameter of the lumen can vary along the longitudinal axis. In some embodiments, the wall thickness of a sleeve can be constant along the longitudinal axis. In some embodiments, the wall thickness of a sleeve can vary along the longitudinal axis. In some embodiments, a perimeter of the sleeve can be constant along the longitudinal axis. In some embodiments, a perimeter of the sleeve can vary along the longitudinal axis. In some embodiments, the lengths of the sleeves can vary. Shorter sleeves are suitable in some embodiments, while longer sleeves are preferred in other embodiments. The sleeve can be rigid in some embodiments. The sleeve can be flexible in some embodiments. The sleeve can be semi-rigid in some embodiments. The sleeve can comprise and/or be formed of a plastic and/or another suitable material for use on a stimulating probe in a body.

In some embodiments, a sleeve can have a tapered shape. For example, the sleeve can taper distally. In some embodiments, at least one advantage of a tapered sleeve includes that the sleeve helps to push tissue away from the probe shaft upon advancement of the sleeve to help dilate tissue. In some embodiments, a plurality of sleeves can be coupled with the probe. The plurality of sleeves can be coupled and/or removed in serial fashion in some cases. The plurality of sleeves can be coupled and/or removed in parallel fashion in some cases. In some embodiments, the probe is disposable. In some embodiments, the probe is reusable. In some embodiments, the sleeve is disposable. In some embodiments, the sleeve is reusable. In some embodiments, the sleeve conforms to the curvature of the stimulating probe. In some embodiments, the stimulating probe is preferably adapted and configured to move coaxially relative to retractor blades forming a channel about an axis. In some embodiments, the sleeve is preferably adapted and configured to move coaxially relative to retractors blades forming a channel about an axis.

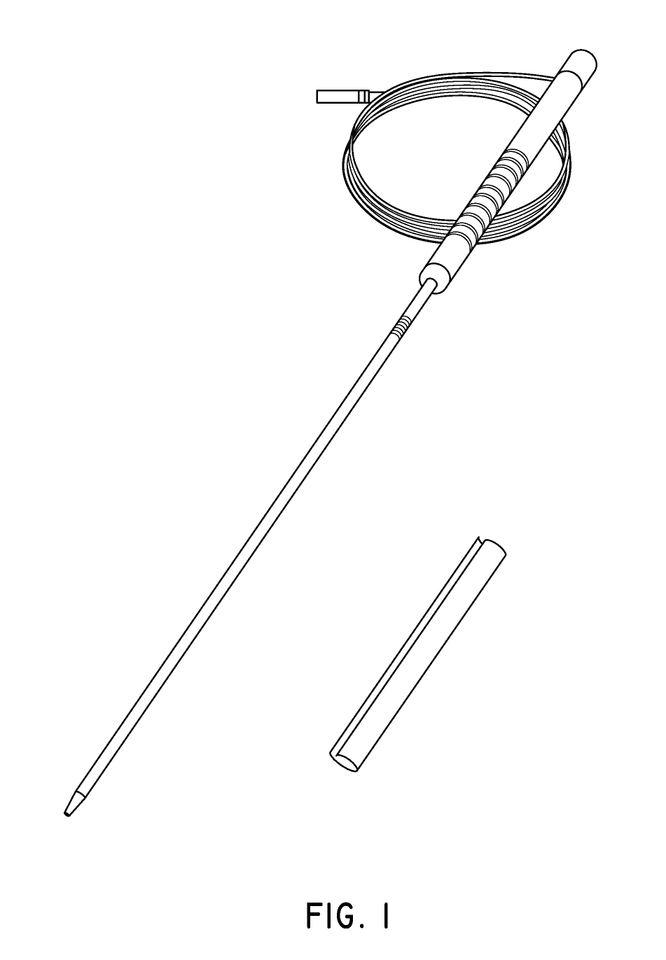

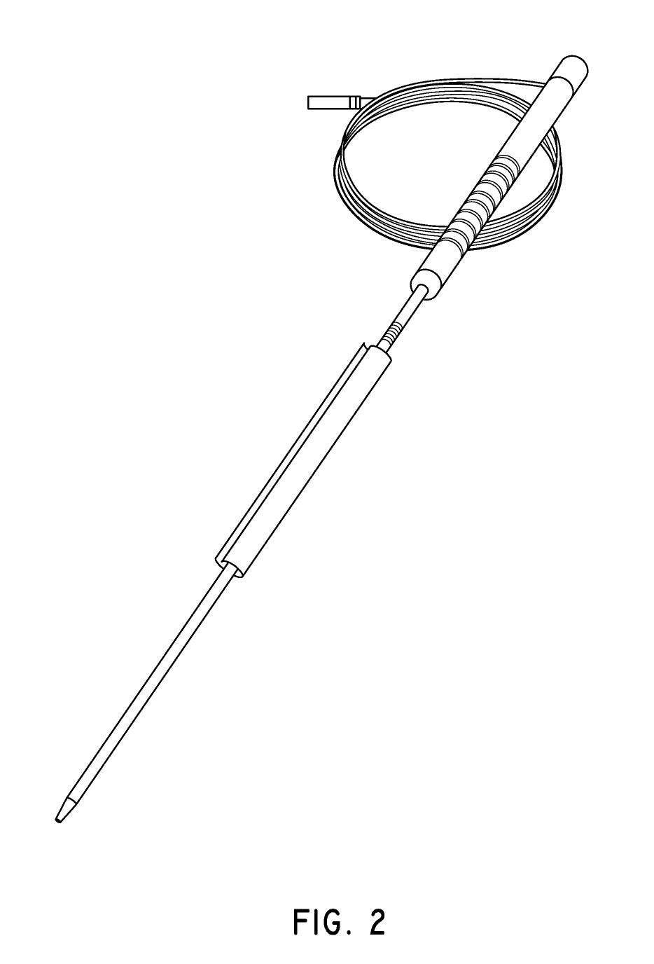

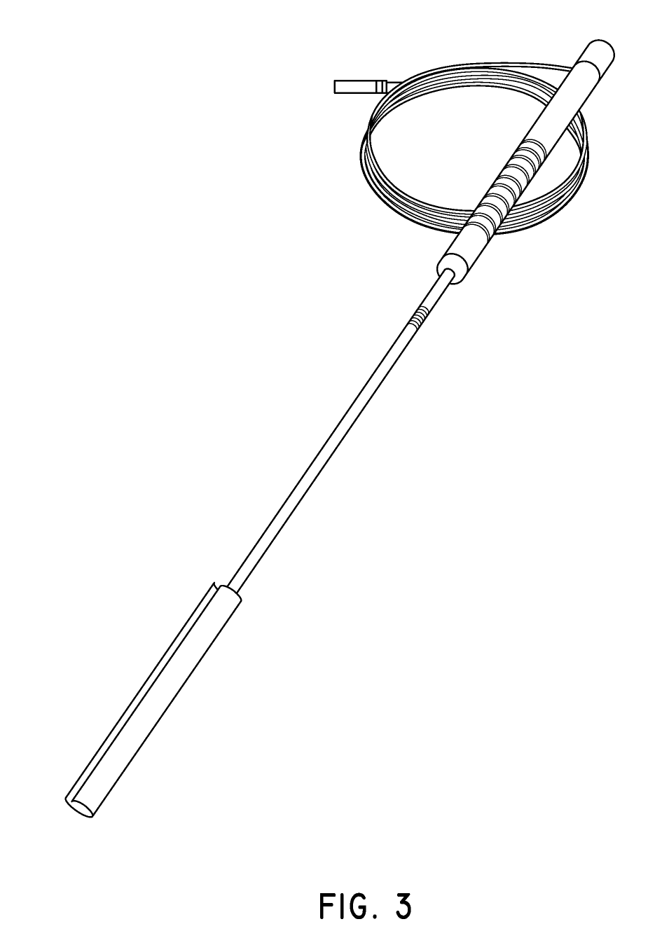

FIGS. 1-3 illustrate an embodiment of at least a portion of a neuromonitoring system that is arranged and configured in accordance with certain features, aspects and advantages of the present disclosure. The illustrated system is similar in some aspects to other systems described herein. The illustrated system shows one embodiment comprising a stimulating probe having an omnidirectional monopoloar electrode tip, and further comprising a sliding, rotating, removable, slotted sleeve configured and adapted to be coupled to the probe. As shown in FIG. 1, the sleeve is preferably a separate part and is detachable from the probe, but is configured to be coupled to the shaft when desired. The sleeve of FIG. 1 comprises a slot that extends the length of the sleeve. The sleeve of FIG. 1 has a uniform cross-section, diameter, and wall thickness. As shown in FIG. 2, the sleeve is coupled to the shaft of the probe in a coaxial fashion, and is rotatable about the shaft and slidable along the longitudinal axis. The sleeve is shown in a proximal, or retracted, or withdrawn configuration, so as not to block omnidirectional signals of the probe tip when in use. As shown in FIG. 3, the sleeve is shown in a distal, or advanced, or shielding configuration, so as to substantially block at least a portion of the distributed signals of the electrode probe tip during use. Preferably a majority of the distribution of signals is blocked in the illustrated configuration. At least a portion of the distributed signals of the electrode probe tip during use preferably passes through the slot in the sleeve. By rotating the sleeve, the surgeon can control the direction of the distributed signal and use the unidirectional features to further navigate during a procedure.

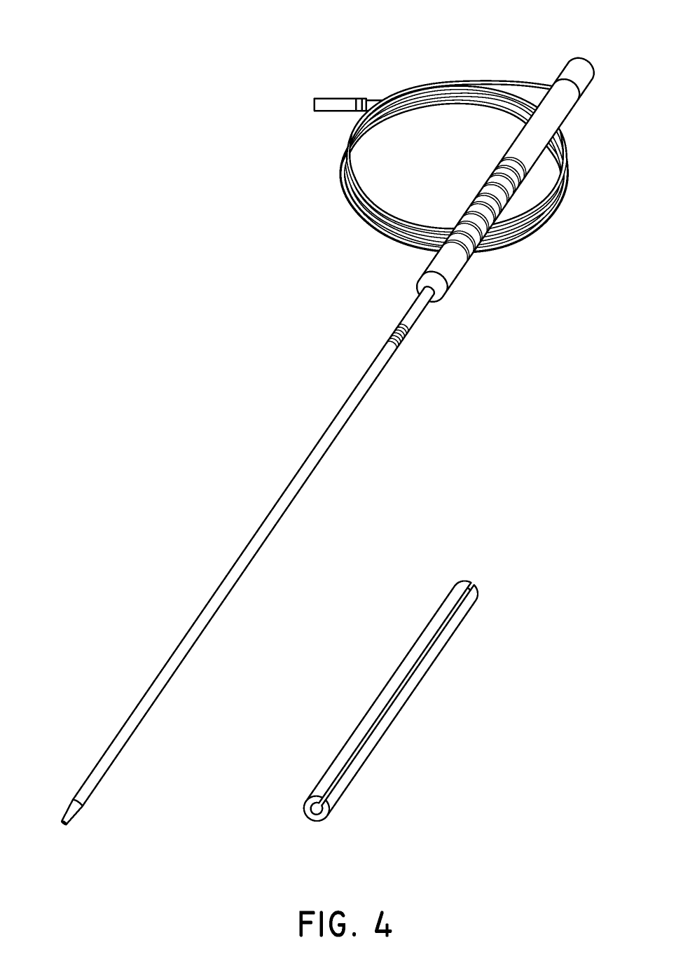

FIG. 4 illustrates an embodiment of at least a portion of a neuromonitoring system that is arranged and configured in accordance with certain features, aspects and advantages of the present disclosure. The illustrated system is similar in some aspects to other systems described herein. The illustrated system shows one embodiment comprising a stimulating probe having an omnidirectional monopoloar electrode tip, and further comprising a sliding, rotating, removable, slotted sleeve configured and adapted to be coupled to the probe. As shown in FIG. 4, the sleeve has a larger wall thickness than the embodiment of FIGS. 1-3.

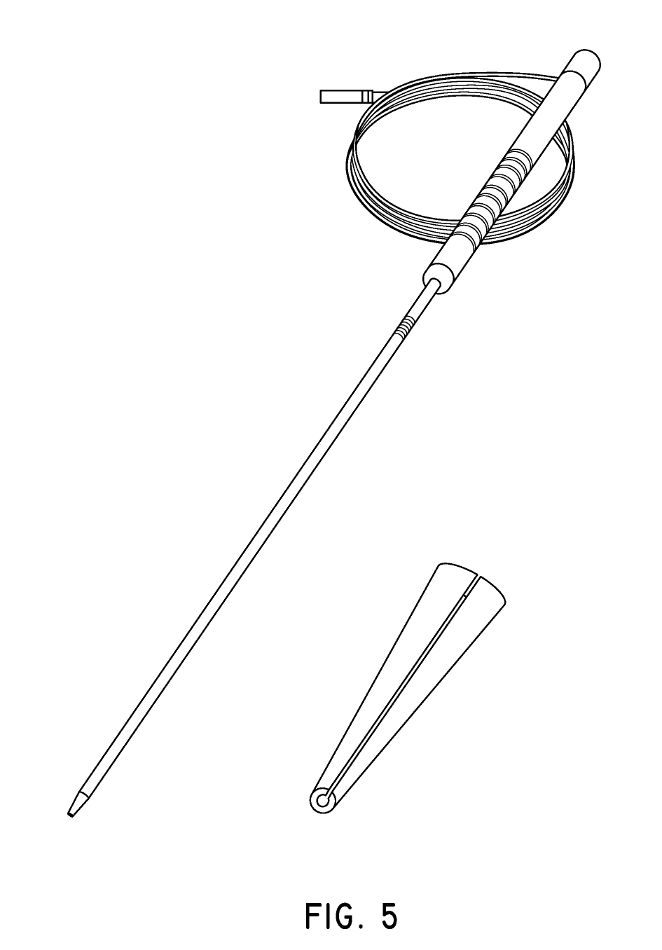

FIG. 5 illustrates an embodiment of at least a portion of a neuromonitoring system that is arranged and configured in accordance with certain features, aspects and advantages of the present disclosure. The illustrated system is similar in some aspects to other systems described herein. The illustrated system shows one embodiment comprising a stimulating probe having an omnidirectional monopoloar electrode tip, and further comprising a sliding, rotating, removable, slotted sleeve configured and adapted to be coupled to the probe. As shown in FIG. 5, the sleeve has a tapered shape. By advancing the sleeve along the longitudinal axis, the surgeon can dilate tissue during a procedure. In some embodiments, the lumen and/or channel has a uniform dimension. In some embodiments, the lumen and/or channel has a tapering configuration.

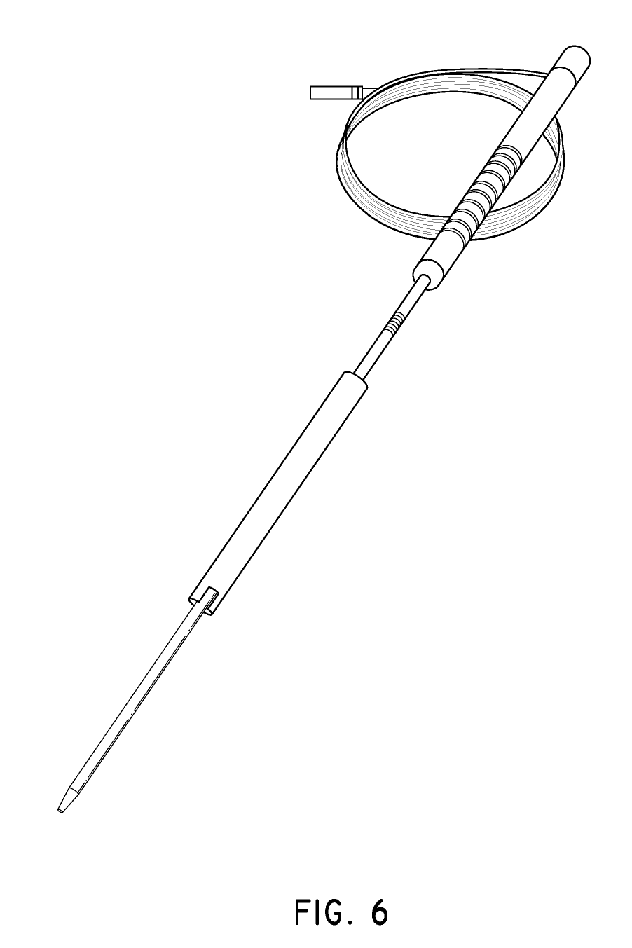

FIG. 6 illustrates an embodiment of at least a portion of a neuromonitoring system that is arranged and configured in accordance with certain features, aspects and advantages of the present disclosure. The illustrated system is similar in some aspects to other systems described herein. The illustrated system shows one embodiment comprising a stimulating probe having an omnidirectional monopoloar electrode tip, and further comprising a sliding, rotating, removable, slotted sleeve configured and adapted to be coupled to the probe. As shown in FIG. 6, the sleeve has a slot that extends only partially along the length thereof. In some embodiments, the sleeve is preferably coupled with the probe shaft prior to when the probe shaft is inserted in a patient. In some embodiments, when the sleeve is configured and adapted to be coaxially movable along the longitudinal axis to a distal, or advanced, or shielding configuration, so as to substantially block at least a portion of the distributed signals of the electrode probe tip during use. Preferably a majority of the distribution of signals is blocked in the illustrated configuration. At least a portion of the distributed signals of the electrode probe tip during use preferably passes through the partial slot in the sleeve. By rotating the sleeve, the surgeon can control the direction of the distributed signal and use the unidirectional features to further navigate during a procedure.

Methods of Use

According to one aspect of the disclosure, a method of using the neuromonitoring system comprises practicing one or more of the following principles and/or steps. In one embodiment, intraoperative neuromonitoring refers to the graphical and acoustic representation as well as the documentation of neurophysiological activity of one or several nerves. An electric stimulation, at a motor peripheral nerve, leads to the formation of action potentials and thus to a contraction of the innervated muscle.

Triggered electromyography (t-EMG) is the form of neuromonitoring where an external stimulus (neuromonitoring probe) is used to generate an action potential, the recording of which in a specific muscle (recording electrodes) identifies the nerve stimulated. t-EMG helps the surgeon in localizing relevant neural structures. By using t-EMG during the lateral approach, the operating time, incision size and tissue dissection can be reduced significantly in some cases.

Neuromonitoring Kits as described herein are specially designed to support triggered EMG for the lateral approach. The monopolar tip allows for stimulation in the surgical field. The isolated shaft along stimulation probe allows stimulation solely at the tip. The sleeves described herein can be used to selectively provide omnidirectional signals and/or unidirectional signals for enhanced guidance and control. The stimulation probe is compatible with lateral eccentric dilators for lateral approach.

During the lateral approach to the spine with a blunt dissection through the psoas muscle, iatrogenic injuries of the nerve roots, the lumbar plexus and/or individual nerves are most likely to occur. Neural injury during this surgery can be caused by compression, stretch, transection and ischemia of nervous structures as well as operative hematoma in the psoas. Using t-EMG during transpsoatic approaches supports the surgeon in the detection of motoric neural structures and in doing so, allows the surgeon to adjusts his/her approach to reduce the occurrence of nerve damage.

A neuromonitoring kit as described herein is intended for use in intraoperative spinal procedures for patient connected intraoperative neuromonitoring where an appropriate neuromonitoring machine is also used. The neuromonitoring kit preferably allows for triggered EMG stimulation and subsequent recording of the stimulus from the muscle whose nerve was initially stimulated. The neuromonitoring kit is advantageous for intraoperative use during lateral approach surgeries where the patient's peripheral motor neural structures are at risk of damage due to manipulation.

In some embodiments, the neuromonitoring stimulation probe and sleeve system comprises a stimulation probe with cable, a reference electrode, and a sleeve. An electrode kit for neuromonitoring preferably comprises a plurality of paired electrodes and at least one ground electrode. A pack preferably is sufficient to monitor at least four muscles. At least one stimulation probe and shied can map nerves in the psoas. If the nerves around the dilators need to be checked in parallel, a second probe can be used. Imaging equipment is preferably available for visualization of instrumentation during the procedure.

A patient is preferably placed in a lateral decubitus position with the iliac crest positioned over the table breaking point and with the preferred side facing upwards. The muscles to be monitored depend on the target operating disc level and access side. In some applications, for recording, one pair of needle electrodes (black and red plugs) is placed in one of the muscles monitored. Two needles of the pair are placed proximal and distal in the same muscle 2-4 cm apart. This procedure is repeated for however many muscles need to be monitored. The set-up is completed with a ground electrode which is placed in the subcutaneous tissue close to the hip.

The placed electrodes are preferably connected to the corresponding input of an intraoperative neurophysiological monitoring system. According to some embodiments, to assemble the stimulation probe with a handle, screw the cable (red) into the handle and push the probe into the handle until it is seated. The reference electrode (black) is placed close to or even in the surgical field and secured with sterile tape. The reference electrode (black) is and the cable of the sterile stimulation probe (red) is preferably connected to the IONM system's output.

The stimulation probe is preferably introduced into the surgical site avoiding any surrounding structures/instruments. According to one application, the probe is advanced with the sleeve uncoupled or retracted such that the probe can generate omnidirectional signals during use. According to one application, the sleeve can then be coupled to the probe and/or advanced longitudinally along the shaft to cover and/or shield at least a portion of the electrode tip such that the tip effectively operates with a unidirectional signal for further navigation and/or advancement as desired. After reaching the psoas muscle, triggered EMG can be used to localize neural structures running through and around the muscle.

In general, a response with a threshold below 5 mA means direct contact with a nerve in the psoas, whereas 5 mA-10 mA indicates a close vicinity to a nerve. Before penetrating the psoas muscle with the stimulation probe, the surface of the muscle is preferably mapped to find the area of highest threshold, which typically resides over the disc level of interest.

If the approach chosen is satisfactory, the probe is slowly inserted through the muscle while stimulating. In some applications the sleeve is uncoupled and/or withdrawn during advancement. In some applications, the sleeve is advanced and at least partially shields the probe tip. During any or all of these manipulations, a warning preferably shows if there is a triggered EMG response at which point appropriate intervention should be taken.

If there is no response from the muscle at stimulation thresholds of above 10 mA, the probe can be advanced into the psoas muscle under lateral and AP fluoroscopy confirming probe placement over the appropriate disc space. The muscle is bluntly dissected with the stimulation probe until it reaches the disc space and anchor it there while checking for neural structures on the way to the disc space.

After bluntly dissecting through the psoas, the surgeon may monitor the circumference of the approach with a second probe to detect nerves in proximity. Once the probe is securely inserted in the vertebral disc, the surgeon can start separating the posas muscle by using the eccentric dilators. The sleeve can be withdrawn and/or decoupled. The handle is preferably removed from the stimulation probe. Steps are preferably taken to keep the access open and perform the necessary procedures.

Although the foregoing description of the preferred embodiments has shown, described and pointed out the fundamental novel features of the invention, it will be understood that various omissions, substitutions, and changes in the form of the detail of the apparatus as illustrated as well as the uses thereof, may be made by those skilled in the art, without departing from the spirit of the invention.

Reference throughout this specification to “one embodiment” or “an embodiment” means that a particular feature, structure or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures or characteristics of any embodiment described above may be combined in any suitable manner, as would be apparent to one of ordinary skill in the art from this disclosure, in one or more embodiments.

Similarly, it should be appreciated that in the above description of embodiments, various features of the inventions are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

What is claimed is:

1. A neuromonitoring system comprising:
a plurality of needle electrodes;
a ground electrode;
a monopolar stimulating probe having an electrode tip and configured to be coupled to a cable; and
a stimulating probe sleeve adapted and configured to be removably coupled to the monopolar stimulating probe, wherein the sleeve has a slot and is configured and adapted to coaxially slide relative to the monopolar stimulating probe between a first retracted position and a second advanced position, wherein the electrode tip generates an omnidirectional signal when in use while the sleeve is in the retracted position or in an uncoupled position, and wherein the electrode tip generates a unidirectional signal when in use while the sleeve is in the advanced position.

2. The system of claim 1, comprising a slot extending an entire length of the sleeve from a proximal portion to a distal portion.

3. The system of claim 1, comprising a slot extending a partial length of the sleeve.

4. The system of claim 1, wherein the sleeve is rotatable about a longitudinal axis of the stimulating probe when coupled.

5. The system of claim 1, wherein the sleeve has a tapered shape.

6. The system of claim 1, wherein the probe is disposable.

7. The system of claim 1, wherein the sleeve is disposable.

8. The system of claim 1, wherein the sleeve conforms to a curvature of the stimulating probe.

9. The system of claim 1, wherein the stimulating probe is adapted and configured to move coaxially relative to retractor blades forming a channel about an axis.

10. The system of claim 1, wherein the sleeve is adapted and configured to move coaxially relative to retractors blades forming a channel about an axis.

11. A method of using a neuromonitoring system, the method comprising:
providing a monopolar stimulating probe having an electrode tip and a stimulating probe sleeve having a slot;
removably coupling the sleeve to the probe; and
coaxially sliding the sleeve relative to the probe to selectively shield an electrode tip of the probe.

12. The method of claim 11, comprising coupling the sleeve to the probe prior to insertion of the probe in a patient.

13. The method of claim 11, comprising coupling the sleeve to the probe after insertion of the probe in a patient.
